(19) United States
(12) Patent Application Publication
(10) Pub. No.: US 2026/0183266 A1
(43) Pub. Date: Jul. 2, 2026

(54) COMPOUNDS FOR TREATING LYMPHOMA OR A T-CELL MALIGNANT DISEASE

(71) Applicant: Purdue Pharma L.P., Stamford, CT (US)

(72) Inventors: Katarina Hilgier, Zurich (CH); Francisco Ibanez Lopez, Alicante (ES); Thomas Jorg Mehrling, Riehen (CH)

(73) Assignee: Purdue Pharma L.P., Stamford, CT (US)

(21) Appl. No.: 19/289,195

(22) Filed: Aug. 4, 2025

Related U.S. Application Data
(63) Continuation of application No. 17/414,797, now Pat. No. 12,377,076.

(30) Foreign Application Priority Data
Dec. 18, 2018 (GB) 1820643.3
Dec. 18, 2018 (GB) 1820645.8
Mar. 6, 2019 (GB) 1903003.0
Mar. 6, 2019 (GB) 1903005.5
Jun. 12, 2019 (GB) 1908434.2
Jun. 12, 2019 (GB) 1908436.7

(51) Int. Cl. A61K 31/4184; A61K 9/00; A61K 45/06; A61P 7/00; A61P 35/00; G16H 20/10

(52) U.S. Cl. CPC A61K 31/4184; A61K 9/0019; A61K 45/06; A61P 7/00; A61P 35/00; G16H 20/10

(57) ABSTRACT

The invention relates to tinostamustine for use in the treatment of lymphoma or a T-cell malignant disease. Tinostamustine may cause side effects when administered to a patient, and it is desirable to minimise such effects. The present invention defines an improved treatment, wherein the dose of tinostamustine administered is varied based on the patient's platelet count.

### REFERENCE TO RELATED APPLICATIONS

This application is a continuation application of U.S. patent application Ser. No. 17/414,797, filed on Jun. 16, 2021, which is a U.S. national stage application filed under 35 U.S.C. § 371, based on International Patent Application No. PCT/IB2019/061034, filed on Dec. 18, 2019, which claims priority to UK Patent Application No. 1820645.8, filed on Dec. 18, 2018, UK Patent Application No. 1820643.3, filed on Dec. 18, 2018, UK Patent Application No. 1903003.0, filed on Mar. 6, 2019, UK Patent Application No. 1903005.5, filed on Mar. 6, 2019, UK Patent Application No. 1908434.2, filed Jun. 12, 2019, and UK Patent Application No. 1908436.7, filed on Jun. 12, 2019. The entire contents of each of the foregoing applications are incorporated herein by reference.

The invention relates to tinostamustine for use in the treatment of lymphoma or a T-cell malignant disease.

### BACKGROUND OF THE INVENTION

Lymphoma is a cancer of the lymphatic system. There are two main types of lymphoma, namely Hodgkin lymphoma and non Hodgkin lymphoma.

Non-Hodgkin lymphoma is the more common form of lymphoma. The lymphatic system runs throughout the body, and it is therefore possible to find non-Hodgkin lymphoma in almost all parts of the body. In patients with non-Hodgkin lymphoma, some of their white blood cells (lymphocytes) divide abnormally. They do not have any resting time like normal cells and they start to divide continuously, so too many are produced which do not naturally die. These cells start to divide before they are fully mature and therefore cannot fight infection as normal white blood cells do. The abnormal lymphocytes start to collect in the lymph nodes or other places such as the bone marrow or spleen. They can then grow into tumours and begin to cause problems within the lymphatic system or the organ in which they are growing. For example, if a lymphoma starts in the thyroid gland it can affect the normal production of thyroid hormones. There are many different types of non-Hodgkin lymphoma. They can be classified in several different ways. One way is by the type of cell affected. In non-Hodgkin lymphoma two types of lymphocyte can be affected—B cells and T cells. This is classified as a B cell lymphoma or a T cell lymphoma. Most people with non-Hodgkin lymphoma have B cell lymphomas. T cell lymphomas are more common in teenagers and young adults.

The cells of Hodgkin lymphoma have a particular appearance under the microscope. These cells are called Reed Sternberg cells. Non Hodgkin lymphomas do not have Reed Sternberg cells. It is important for doctors to be able to tell the difference between Hodgkin lymphoma and non-Hodgkin lymphoma cells as they are two different diseases. In Hodgkin lymphoma, it is cells in the lymph nodes that have become cancerous.

The % survival rate over five years in 2009 for patients with non-Hodgkin lymphoma was 63%, while the survival rate for those with Hodgkin lymphoma over the same period was 83%.

T-cell malignant diseases include T-cell prolymphocytic leukemia, which is a rare and unusual malignancy characterized by the proliferation of small- to medium-sized prolymphocytes of postthymic origin with distinctive clinical, morphologic, immunophenotypic, and cytogenetic features. Involvement of the peripheral blood, bone marrow, lymph nodes, liver, spleen, and skin can occur. The clinical course is typically very aggressive with poor response to conventional chemotherapy and short survival rates.

Chemotherapy involves the disruption of cell replication or cell metabolism. However, because of the difficulty in targeting the cancer cells specifically, chemotherapy often causes serious toxic adverse effects. Accordingly, there is a need for more effective cancer treatments, in particular for the treatment of lymphoma. In particular, there is a need for methods of treatment which use the highest possible dose of the chemotherapeutic and therefore provide the maximum therapeutic benefit. However, advantageously, this dose should be tailored, and not exceed the maximum dose that can be tolerated by the particular patient.

WO 2010/085377 discloses tinostamustine (or EDO-S101), which is a first-in-class alkylating deacetylase inhibiting molecule:

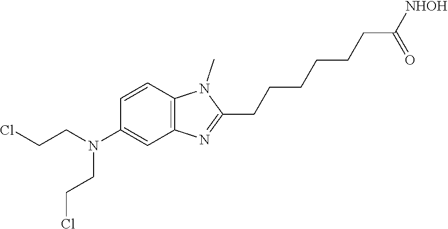

Tinostamustine has shown potent activity in in vitro and in vivo models against lymphoma (see The First-In-Class Alkylating Histone Deacetylase Inhibitor (HDACi) Fusion Molecule EDO-S101 Exerts Potent Preclinical Activity Against Tumor Cells of Hodgkin Lymphoma (HL) Including Bendamustine Resistant Clones, Di Filippi R et al, 57th Annual Meeting and Exposition of the American Society of Hematology (ASH), 6 Dec. 2015.).

### SUMMARY OF THE INVENTION

In a first aspect the present invention provides tinostamustine or a pharmaceutically acceptable salt thereof for use in the treatment of lymphoma or a T-cell malignant disease in a patient, wherein the treatment involves:
(a) determining whether the patient's initial platelet count is equal to or above a first value, equal to or below a second value, or between the first and second values, wherein the first value is greater than the second value; and
(b) if the initial platelet count is above the first value then administering a first amount of the tinostamustine or pharmaceutically acceptable salt thereof to the patient, if the initial platelet count is equal to or below the second value then administering a second amount of the tinostamustine, and if the initial platelet count is between the first and second values then administering a third amount of the tinostamustine, wherein the first amount is greater than the third amount and the third amount is greater than the second amount.

In a first aspect the present invention also provides a use of tinostamustine or a pharmaceutically acceptable salt thereof for the manufacture of a medicament for use in the treatment of lymphoma or a T-cell malignant disease in a patient, wherein the treatment involves:
(a) determining whether the patient's initial platelet count is above or below a particular value; and
(b) if the initial platelet count is above the particular value then administering a first amount of the tinostamustine or pharmaceutically acceptable salt thereof to the patient, and if the initial platelet count is below the particular value then administering a second amount of the tinostamustine or pharmaceutically acceptable salt thereof to the patient, wherein the first amount is greater than the second amount.

In a first aspect the present invention also provides a method of treating lymphoma or a T-cell malignant disease in a patient comprising the steps of:
(a) determining whether the patient's initial platelet count is above or below a particular value; and
(b) if the initial platelet count is above the particular value then administering a first amount of tinostamustine or a pharmaceutically acceptable salt thereof to the patient, and if the initial platelet count is below the particular value then administering a second amount of tinostamustine or pharmaceutically acceptable salt thereof to the patient, wherein the first amount is greater than the second amount.

In a second aspect, the present invention provides tinostamustine or a pharmaceutically acceptable salt thereof for use in the treatment of lymphoma or a T-cell malignant disease in a patient, wherein the patient has a baseline platelet count of greater than or equal to 200×10⁹/L and the tinostamustine or pharmaceutically acceptable salt thereof is administered at a dose of 95 mg/m² or greater (for example 95-105 mg/m² e.g. 100 mg/m²) based on free tinostamustine and the patient's body surface area.

In a second aspect, the present invention also provides a use of tinostamustine or a pharmaceutically acceptable salt thereof for the manufacture of a medicament for use in the treatment of lymphoma or a T-cell malignant disease in a patient, wherein the patient has a baseline platelet count of greater than or equal to 200×10⁹/L and the tinostamustine or pharmaceutically acceptable salt thereof is administered at a dose of 95 mg/m² or greater (for example 95-105 mg/m² e.g. 100 mg/m²) based on free tinostamustine and the patient's body surface area.

In a second aspect, the present invention also provides a method of treating lymphoma or a T-cell malignant disease in a patient wherein the patient has a baseline platelet count of greater than or equal to 200×10⁹/L, comprising the step of administering to the patient tinostamustine or a pharmaceutically acceptable salt thereof at a dose of 95 mg/m² or greater (for example 95-105 mg/m² e.g. 100 mg/m²) based on free tinostamustine and the patient's body surface area.

In a third aspect, the present invention provides tinostamustine or a pharmaceutically acceptable salt thereof for use in the treatment of lymphoma or a T-cell malignant disease in a patient, wherein the patient has a baseline platelet count of between 100×10⁹/L and 200×10⁹/L and the tinostamustine or pharmaceutically acceptable salt thereof is administered at a dose of 75-85 mg/m² (for example 80 mg/m²) based on free tinostamustine and the patient's body surface area.

In a third aspect, the present invention also provides the use of tinostamustine or a pharmaceutically acceptable salt thereof for the manufacture of a medicament for use in the treatment of lymphoma or a T-cell malignant disease in a patient, wherein the patient has a baseline platelet count of between 100×10⁹/L and 200×10⁹/L and the tinostamustine or pharmaceutically acceptable salt thereof is administered at a dose of 75-85 mg/m² (for example 80 mg/m²) based on free tinostamustine and the patient's body surface area.

In a third aspect, the present invention provides a method of treating lymphoma or a T-cell malignant disease in a patient wherein the patient has a baseline platelet count of between 100×10⁹/L and 200×10⁹/L, comprising the step of administering to the patient tinostamustine or a pharmaceutically acceptable salt thereof at a dose of 75-85 mg/m² (for example 80 mg/m²) based on free tinostamustine and the patient's body surface area.

In a fourth aspect, the present invention provides tinostamustine or a pharmaceutically acceptable salt thereof for use in the treatment of lymphoma or a T-cell malignant disease in a patient, wherein the patient has a baseline platelet count of less than or equal to 100×10⁹/L and the tinostamustine or pharmaceutically acceptable salt thereof is administered at a dose of 54 mg/m² or lower (for example 45-54 mg/m² e.g. 50 mg/m²) based on free tinostamustine and the patient's body surface area.

In a fourth aspect, the present invention also provides a use of tinostamustine or a pharmaceutically acceptable salt thereof for the manufacture of a medicament for use in the treatment of lymphoma or a T-cell malignant disease in a patient, wherein the patient has a baseline platelet count of less than or equal to 100×10⁹/L and the tinostamustine or pharmaceutically acceptable salt thereof is administered at a dose of 54 mg/m² or lower (for example 45-54 mg/m² e.g. 50 mg/m²) based on free tinostamustine and the patient's body surface area.

In a fourth aspect, the present invention also provides a method of treating lymphoma or a T-cell malignant disease in a patient wherein the patient has a baseline platelet count of less than or equal to 100×10⁹/L, comprising the step of administering to the patient tinostamustine or a pharmaceutically acceptable salt thereof at a dose 54 mg/m² or lower (for example 45-54 mg/m² e.g. 50 mg/m²) based on free tinostamustine and the patient's body surface area.

In a fifth aspect, the present invention provides tinostamustine or a pharmaceutically acceptable salt thereof for use in the treatment of lymphoma or a T-cell malignant disease in a patient, wherein the tinostamustine or pharmaceutically acceptable salt thereof is administered:
intravenously;
at a dose of from 20-100 mg/m² based on free tinostamustine and the patient's body surface area, for example 95-105 mg/m², 75-85 mg/m², 55-65 mg/m², 45-55 mg/m² or 35-45 mg/m² (e.g. 100 mg/m²; 80 mg/m², 60 mg/m², 50 mg/m² or 40 mg/m²); and
over a period of time which is 45-75 minutes, for example 50-70 minutes or 55-65 minutes e.g. 60 minutes.

In a fifth aspect, the present invention provides tinostamustine or a pharmaceutically acceptable salt thereof for the manufacture of a medicament for use in the treatment of lymphoma or a T-cell malignant disease in a patient, wherein the tinostamustine or pharmaceutically acceptable salt thereof is administered:
intravenously;
at a dose of from 20-100 mg/m² based on free tinostamustine and the patient's body surface area, for example 95-105 mg/m², 75-85 mg/m², 55-65 mg/m², 45-55 mg/m² or 35-45 mg/m² (e.g. 100 mg/m²; 80 mg/m², 60 mg/m², 50 mg/m² or 40 mg/m²); and
over a period of time which is 45-75 minutes, for example 50-70 minutes or 55-65 minutes e.g. 60 minutes.

In a fifth aspect, the present invention also provides a method of treating lymphoma or a T-cell malignant disease in a patient comprising the step of administering to the patient tinostamustine or a pharmaceutically acceptable salt thereof:
intravenously;
at a dose of from 20-100 mg/m² based on free tinostamustine and the patient's body surface area, for example 95-105 mg/m², 75-85 mg/m², 55-65 mg/m², 45-55 mg/m² or 35-45 mg/m² (e.g. 100 mg/m²; 80 mg/m², 60 mg/m², 50 mg/m² or 40 mg/m²); and
over a period of time which is 45-75 minutes, for example 50-70 minutes or 55-65 minutes e.g. 60 minutes.

### BRIEF DESCRIPTION OF THE FIGURES

The invention will be described with reference to the following figures, wherein:

FIG. 1 shows the day one median PK profiles of tinostamustine by dose group;

FIG. 2 shows the individual day one tinostamustine Cₘₐₓ versus AUC_{0-8 hours} linear regression fit;

FIG. 3 shows the relative effect on lymphocyte (LYM), neutrophil (NEU) or platelet (PLT) counts versus dose from the exploratory analysis;

FIG. 4 shows the model fit of the blood cell counts;

FIG. 5 shows the simulated dose-nadir relationships for patients with platelet cell counts ranging from 50-200 and 200-450 10⁹/L;

FIG. 6 shows a boxplot showing the median, 25% and 75% percentile of platelet counts at baseline;

FIG. 7 is a cumulative curve of platelet count at baseline and respective proportion of patients per group;

FIG. 8 is a boxplot of median, 25% and 75% percentile platelet counts at baseline per underlying disease; and

FIG. 9 shows the reference dose for 20% likely grade 4 thrombocytopenia and 90% grade 3 lymphocytopenia.

### DEFINITIONS

### Dose of Tinostamustine

The dose of tinostamustine is defined herein by reference to free tinostamustine. The term free tinostamustine means tinostamustine that is not in the form of a pharmaceutically acceptable salt. In the event that the invention is implemented using a salt of tinostamustine, then the mass of the salt administered is adjusted to provide the same number of moles of tinostamustine as is present in the masses of free tinostamustine defined herein.

### Patient Surface Area

The dose of tinostamustine is defined herein by reference to amount of free tinostamustine used relative to the patient's surface area i.e. mg/m². The skilled clinician is able to calculate the patient surface area using the common general knowledge.

In particular, patient surface area (PSA) can be calculated using the following formula (Dubois D, Dubois EF, A formula to estimate the approximate surface area if height and weight be known, Arch Intern Med, 1916, 17, 863-871):

$$PSA = 0.007184 \times {( patient height in cm )}^{0.725} \times {( patient weight in kg )}^{0.425}$$

### Platelet Count

Platelets, also known as thrombocytes, are a component of blood that reacts to bleeding by clumping and thereby initiating a blood clot. The platelet count of a patient can be determined by part of a routine complete blood count in which a sample of blood is taken and then analysed to give the number of platelets per litre of blood.

The platelet count can be measured in two ways, namely a manual visual method and an automated electronic method.

A visual platelet count may be determined manually using a hemocytometer, where the number of platelets can be counted in a specific volume of blood.

An electronic method uses an automated blood cell analyser (e.g. a Coulter S-Plus) which counts particles in the bloodstream. However, for very low counts e.g. below 50×10⁹/L, an electronic measurement may not be accurate and should be confirmed by a manual count.

A low platelet concentration (less than 150×10⁹/L) is known as thrombocytopenia and can be a result of decreased production or depletion.

### Claim Format

In the general aspects of invention described above, the invention is described using the European compound-for-use medical use format, the Swiss-format and method of treatment format. For the sake of conciseness, in the following detailed description we have defined the invention using only the compound-for-use format. However, these passages should also be considered to further define the invention in the method of treatment format and Swiss-format.

The invention is defined herein by reference to the treatment of the disease. The term treatment should be interpreted to cover prophylaxis too, by which is meant in preventing occurrence of a disease or guarding from a disease. Prophylaxis includes complete and total blocking of all symptoms of a disorder for an indefinite period of time, the mere slowing of the onset of one or several symptoms of the disease, or making the disease less likely to occur.

### DETAILED DESCRIPTION OF THE INVENTION

The present invention relates to tinostamustine or a pharmaceutically acceptable salt thereof for use in the treatment of lymphoma or a T-cell malignant disease.

As discussed, it is desirable to calculate the maximum dose that can be tolerated by a particular patient. This allows each patient to obtain the maximum possible therapeutic benefit while minimising the risk of unacceptable and possibly fatal adverse chemotherapeutic effects. Prior to performing the phase 1 human trial, the skilled person would not have predicted what adverse effects would limit the maximum tolerated dose. Early studies suggested that the dose may be limited by cardiac safety.

However, surprisingly, the applicant found that thrombocytopenia was dose limiting. Further, the applicant discovered that the probability of developing thrombocytopenia could be predicted solely by the patient's baseline platelet count. Accordingly, the invention provides an improved treatment, wherein the maximum dose that can be tolerated by a particular patient can be calculated by measuring their platelet count prior to therapy, in order to provide the best possible balance between therapeutic and adverse effects.

Accordingly, in a first aspect the present invention provides tinostamustine or a pharmaceutically acceptable salt thereof for use in the treatment of lymphoma or a T-cell malignant disease in a patient, wherein the treatment involves:
(a) determining whether the patient's initial platelet count is equal to or above a first value, equal to or below a second value, or between the first and second values, wherein the first value is greater than the second value; and
(b) if the initial platelet count is above the first value then administering a first amount of the tinostamustine or pharmaceutically acceptable salt thereof to the patient, if the initial platelet count is equal to or below the second value then administering a second amount of the tinostamustine, and if the initial platelet count is between the first and second values then administering a third amount of the tinostamustine, wherein the first amount is greater than the third amount and the third amount is greater than the second amount.

In particular, the patient is a human.

Thus, prior to administration, the patient to be treated has their platelet count measured, and based on the platelet count the appropriate amount of tinostamustine to be administered is determined. In particular, if a patient has a higher platelet count, then the patient can tolerate a higher dose of tinostamustine. If a patient has a lower platelet count, a lower dose of tinostamustine can be tolerated.

In particular embodiments, the patient's initial platelet count is determined to be equal to or above a first value which is:
between 165×10⁹/L and 175×10⁹/L;
between 170×10⁹/L and 180×10⁹/L;
between 175×10⁹/L and 185×10⁹/L;
between 180×10⁹/L and 190×10⁹/L;
between 190×10⁹/L and 200×10⁹/L;
between 195×10⁹/L and 205×10⁹/L;
between 200×10⁹/L and 210×10⁹/L;
between 205×10⁹/L and 215×10⁹/L;
between 210×10⁹/L and 220×10⁹/L; and
between 215×10⁹/L and 225×10⁹/L.

Preferably, the patient's initial platelet count is determined to be equal to or above a first value which is 200×10⁹/L.

In other embodiments, the patient's initial platelet count is determined to be equal to or above a first value which is:
170×10⁹/L;
175×10⁹/L;
180×10⁹/L;
185×10⁹/L;
190×10⁹/L;
195×10⁹/L;
205×10⁹/L;
210×10⁹/L;
215×10⁹/L; or
220×10⁹/L.

Preferably, if the patient's platelet count is determined to be equal to or above the first value, then the amount of tinostamustine or pharmaceutically acceptable salt thereof is 95 mg/m² or greater (for example 95-105 mg/m² e.g. 100 mg/m²) based on free tinostamustine and the patient's body surface area. In particular, if the patient's platelet count is determined to be over the relevant value, then the amount of tinostamustine or pharmaceutically acceptable salt thereof is 100 mg/m² based on free tinostamustine and the patient's body surface area.

In other embodiments, if the patient's platelet count is determined to be over the relevant value, then the amount of tinostamustine or pharmaceutically acceptable salt thereof is:
from 75 to 85 mg/m² (e.g. 80 mg/m²) based on free tinostamustine and the patient's body surface area;
from 80 to 90 mg/m² (e.g. 85 mg/m²) based on free tinostamustine and the patient's body surface area;
from 85 to 105 mg/m² (e.g. 90 mg/m²) based on free tinostamustine and the patient's body surface area;
from 90 to 100 mg/m² (e.g. 95 mg/m²) based on free tinostamustine and the patient's body surface area;
from 100 to 110 mg/m² (e.g. 105 mg/m²) based on free tinostamustine and the patient's body surface area;
from 105 to 115 mg/m² (e.g. 110 mg/m²) based on free tinostamustine and the patient's body surface area;
from 110 to 120 mg/m² (e.g. 115 mg/m²) based on free tinostamustine and the patient's body surface area; or
from 115 to 125 mg/m² (e.g. 120 mg/m²) based on free tinostamustine and the patient's body surface area.

In particular embodiments, the patient's initial platelet count is determined to be equal to or below a second value which is:
between 65×10⁹/L and 75×10⁹/L;
between 70×10⁹/L and 80×10⁹/L;
between 75×10⁹/L and 85×10⁹/L;
between 80×10⁹/L and 90×10⁹/L;
between 90×10⁹/L and 100×10⁹/L;
between 95×10⁹/L and 105×10⁹/L;
between 100×10⁹/L and 110×10⁹/L;
between 105×10⁹/L and 115×10⁹/L;
between 110×10⁹/L and 120×10⁹/L; and
between 115×10⁹/L and 125×10⁹/L.

Preferably, the patient's initial platelet count is determined to be equal to or below a second value which is 100×10⁹/L.

In other embodiments, the patient's initial platelet count is determined to be equal to or below a second value which is:
70×10⁹/L;
75×10⁹/L;
80×10⁹/L;
85×10⁹/L;
90×10⁹/L;
95×10⁹/L
105×10⁹/L;
110×10⁹/L;
115×10⁹/L; or
120×10⁹/L.

Preferably, if the patient's platelet count is determined to be equal to or below the second value, then the amount of tinostamustine or pharmaceutically acceptable salt thereof is 54 mg/m² or lower (for example 45-54 mg/m² e.g. 50 mg/m²) based on free tinostamustine and the patient's body surface area. In particular, if the patient's platelet count is determined to be over the relevant value, then the amount of tinostamustine or pharmaceutically acceptable salt thereof is 50 mg/m² based on free tinostamustine and the patient's body surface area.

In other embodiments, if the patient's platelet count is determined to be equal to or below the second value, then the amount of tinostamustine or pharmaceutically acceptable salt thereof is:
from 25 to 35 mg/m² (e.g. 30 mg/m²) based on free tinostamustine and the patient's body surface area;
from 30 to 40 mg/m² (e.g. 35 mg/m²) based on free tinostamustine and the patient's body surface area;
from 35 to 45 mg/m² (e.g. 40 mg/m²) based on free tinostamustine and the patient's body surface area;
from 40 to 50 mg/m² (e.g. 45 mg/m²) based on free tinostamustine and the patient's body surface area;
from 50 to 60 mg/m² (e.g. 55 mg/m²) based on free tinostamustine and the patient's body surface area;
from 55 to 65 mg/m² (e.g. 60 mg/m²) based on free tinostamustine and the patient's body surface area;
from 60 to 70 mg/m² (e.g. 65 mg/m²) based on free tinostamustine and the patient's body surface area; or
from 65 to 75 mg/m² (e.g. 70 mg/m²) based on free tinostamustine and the patient's body surface area.

In particular embodiments, the patient's initial platelet count is determined to be within a range defined by first and second values which is:
75-175×10⁹/L;
80-180×10⁹/L;
85-185×10⁹/L;
90-100×10⁹/L;
95-195×10⁹/L;
105-205×10⁹/L;
110-210×10⁹/L;
115-215×10⁹/L; or
120-220×10⁹/L.

Preferably, the patient's initial platelet count is determined to be within a range defined by first and second values which is 100-200×10⁹/L.

Preferably, if the patient's platelet count is determined to be within a range defined by first and second values, then the amount of tinostamustine or pharmaceutically acceptable salt thereof is 75-85 mg/m² (for example 80 mg/m²) based on free tinostamustine and the patient's body surface area. In particular, if the patient's platelet count is determined to be within a range defined by first and second values, then the amount of tinostamustine or pharmaceutically acceptable salt thereof is 80 mg/m² based on free tinostamustine and the patient's body surface area.

In other embodiments, if the patient's platelet count is determined to be within a range defined by first and second values, then the amount of tinostamustine or pharmaceutically acceptable salt thereof is:
from 55 to 65 mg/m² (e.g. 60 mg/m²) based on free tinostamustine and the patient's body surface area;
from 60 to 70 mg/m² (e.g. 65 mg/m²) based on free tinostamustine and the patient's body surface area;
from 65 to 75 mg/m² (e.g. 70 mg/m²) based on free tinostamustine and the patient's body surface area;
from 70 to 80 mg/m² (e.g. 75 mg/m²) based on free tinostamustine and the patient's body surface area;
from 80 to 90 mg/m² (e.g. 85 mg/m²) based on free tinostamustine and the patient's body surface area;
from 85 to 95 mg/m² (e.g. 90 mg/m²) based on free tinostamustine and the patient's body surface area;
from 90 to 100 mg/m² (e.g. 95 mg/m²) based on free tinostamustine and the patient's body surface area; or
from 95 to 105 mg/m² (e.g. 100 mg/m²) based on free tinostamustine and the patient's body surface area.

Accordingly, the invention provides tinostamustine or a pharmaceutically acceptable salt thereof for use in the treatment of lymphoma or a T-cell malignant disease in a patient wherein:
(i) if the patient's initial platelet count is greater than or equal to 200×10⁹/L then the amount of tinostamustine or pharmaceutically acceptable salt thereof is 95 mg/m² or greater (for example 95-105 mg/m² e.g. 100 mg/m²) based on free tinostamustine and the patient's body surface area; and
(ii) if the patient's initial platelet count is less than or equal to 100×10⁹/L then the amount of tinostamustine or pharmaceutically acceptable salt thereof is 54 mg/m² or lower (for example 45-54 mg/m² e.g. 50 mg/m²) based on free tinostamustine and the patient's body surface area; and
(iii) if the patient's initial platelet count is between 100×10⁹/L and 200×10⁹/L then the amount of tinostamustine or pharmaceutically acceptable salt thereof is 75-85 mg/m² (for example 80 mg/m²) based on free tinostamustine and the patient's body surface area.

In particular, the invention provides tinostamustine or a pharmaceutically acceptable salt thereof for use in the treatment of lymphoma or a T-cell malignant disease in a patient wherein:
(i) if the patient's initial platelet count is greater than or equal to 200×10⁹/L then the amount of tinostamustine or pharmaceutically acceptable salt thereof is 100 mg/m² based on free tinostamustine and the patient's body surface area; and
(ii) if the patient's initial platelet count is less than or equal to 100×10⁹/L then the amount of tinostamustine or pharmaceutically acceptable salt thereof is 50 mg/m² based on free tinostamustine and the patient's body surface area; and
(iii) if the patient's initial platelet count is between 100×10⁹/L and 200×10⁹/L then the amount of tinostamustine or pharmaceutically acceptable salt thereof is 80 mg/m² based on free tinostamustine and the patient's body surface area.

In a second aspect, the present invention provides tinostamustine or a pharmaceutically acceptable salt thereof for use in the treatment of lymphoma or a T-cell malignant disease in a patient, wherein the patient has a baseline platelet count of greater than or equal to 200×10⁹/L and the tinostamustine or pharmaceutically acceptable salt thereof is administered at a dose of 95 mg/m² or greater (for example 95-105 mg/m² e.g. 100 mg/m²) based on free tinostamustine and the patient's body surface area.

In particular, the invention provides tinostamustine or a pharmaceutically acceptable salt thereof for use in the treatment of lymphoma or a T-cell malignant disease in a patient, wherein the patient has a baseline platelet count of greater than or equal to 200×10⁹/L and the tinostamustine or pharmaceutically acceptable salt thereof is administered at a dose of 100 mg/m² based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count equal to or above a first value which is:
170×10⁹/L;
175×10⁹/L;
180×10⁹/L;
185×10⁹/L;
190×10⁹/L;
195×10⁹/L;
205×10⁹/L;
210×10⁹/L;
215×10⁹/L; or
220×10⁹/L;
   and the tinostamustine or pharmaceutically acceptable salt thereof is administered at a dose of 95 mg/m² or greater (for example 95-105 mg/m² e.g. 100 mg/m²) based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count of equal to or greater than 200×10⁹/L and the tinostamustine or pharmaceutically acceptable salt thereof is administered at a dose of:
80 mg/m² based on free tinostamustine and the patient's body surface area;
85 mg/m² based on free tinostamustine and the patient's body surface area;
90 mg/m² based on free tinostamustine and the patient's body surface area;
95 mg/m² based on free tinostamustine and the patient's body surface area;
105 mg/m² based on free tinostamustine and the patient's body surface area;
110 mg/m² based on free tinostamustine and the patient's body surface area;
115 mg/m² based on free tinostamustine and the patient's body surface area;
120 mg/m² based on free tinostamustine and the patient's body surface area.

Other possible combinations of platelet levels and doses are as follows:

| | | Dose based on free tinostamustine (mg/m²) | | | | | | | |
|---|---|---|---|---|---|---|---|---|---|
| | | 75-85 | 80-90 | 85-95 | 90-100 | 95-105 | 100-110 | 105-115 | 110-120 |
| Platelet level | 170 | A1 | B1 | C1 | D1 | E1 | F1 | G1 | H1 |
| greater than or | 175 | A2 | B2 | C2 | D2 | E2 | F2 | G2 | H2 |
| equal to | 180 | A3 | B3 | C3 | D3 | E3 | F3 | G3 | H3 |
| (×10⁹/L) | 185 | A4 | B4 | C4 | D4 | E4 | F4 | G4 | H4 |
| | 190 | A5 | B5 | C5 | D5 | E5 | F5 | G5 | H5 |
| | 195 | A6 | B6 | C6 | D6 | E6 | F6 | G6 | H6 |
| | 200 | A7 | B7 | C7 | D7 | E7 | F7 | G7 | H7 |
| | 205 | A8 | B8 | C8 | D8 | E8 | F8 | G8 | H8 |
| | 210 | A9 | B9 | C9 | D9 | E9 | F9 | G9 | H9 |
| | 215 | A10 | B10 | C10 | D10 | E10 | F10 | G10 | H10 |
| | 220 | A11 | B11 | C11 | D11 | E11 | F11 | G11 | H11 |

In other particular embodiments, the invention provides tinostamustine or a pharmaceutically acceptable salt thereof for use in the treatment of lymphoma or a T-cell malignant disease in a patient, wherein the patient baseline platelet count and the dose of tinostamustine or pharmaceutically acceptable salt thereof is selected from anyone of A1-A11, B1-B11, C1-C11, D1-D11, E1-E11, F1-F11, G1-G11 or H1-H11 as defined in the table above.

In a third aspect, the present invention provides tinostamustine or a pharmaceutically acceptable salt thereof for use in the treatment of lymphoma or a T-cell malignant disease in a patient, wherein the patient has a baseline platelet count of between 100×10⁹/L and 200×10⁹/L and the tinostamustine or pharmaceutically acceptable salt thereof is administered at a dose of 75-85 mg/m² (for example 80 mg/m²) based on free tinostamustine and the patient's body surface area.

In particular, the invention provides tinostamustine or a pharmaceutically acceptable salt thereof for use in the treatment of lymphoma or a T-cell malignant disease in a patient, wherein the patient has a baseline platelet count of between 100×10⁹/L and 200×10⁹/L and the tinostamustine or pharmaceutically acceptable salt thereof is administered at a dose of 80 mg/m² based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count which is:
75-175×10⁹/L;
80-180×10⁹/L;
85-185×10⁹/L;
90-100×10⁹/L;
95-195×10⁹/L;
105-205×10⁹/L;
110-210×10⁹/L;
115-215×10⁹/L; or
120-220×10⁹/L;
   and the tinostamustine or pharmaceutically acceptable salt thereof is administered at a dose of 75-85 mg/m² (for example 80 mg/m²) based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count of between 100×10⁹/L and 200×10⁹/L and the tinostamustine or pharmaceutically acceptable salt thereof is administered at a dose of:
60 mg/m² based on free tinostamustine and the patient's body surface area;
65 mg/m² based on free tinostamustine and the patient's body surface area;
70 mg/m² based on free tinostamustine and the patient's body surface area;
75 mg/m² based on free tinostamustine and the patient's body surface area;
85 mg/m² based on free tinostamustine and the patient's body surface area;
90 mg/m² based on free tinostamustine and the patient's body surface area;
95 mg/m² based on free tinostamustine and the patient's body surface area; or
700 mg/m² based on free tinostamustine and the patient's body surface area.

Other possible combinations of platelet levels and doses are as follows:

| | | Dose based on free tinostamustine (mg/m²) | | | | | | | |
|---|---|---|---|---|---|---|---|---|---|
| | | 30-40 | 35-45 | 40-50 | 45-55 | 50-60 | 55-65 | 60-70 | 65-75 |
| Platelet | 75-175 | A1 | B1 | C1 | D1 | E1 | F1 | G1 | H1 |
| level | 80-180 | A2 | B2 | C2 | D2 | E2 | F2 | G2 | H2 |
| between | 85-185 | A3 | B3 | C3 | D3 | E3 | F3 | G3 | H3 |
| (×10⁹/L) | 90-190 | A4 | B4 | C4 | D4 | E4 | F4 | G4 | H4 |
| | 95-195 | A5 | B5 | C5 | D5 | E5 | F5 | G5 | H5 |
| | 100-200 | A6 | B6 | C6 | D6 | E6 | F6 | G6 | H6 |
| | 105-205 | A7 | B7 | C7 | D7 | E7 | F7 | G7 | H7 |
| | 110-210 | A8 | B8 | C8 | D8 | E8 | F8 | G8 | H8 |
| | 115-215 | A9 | B9 | C9 | D9 | E9 | F9 | G9 | H9 |
| | 120-220 | A10 | B10 | C10 | D10 | E10 | F10 | G10 | H10 |

In other particular embodiments, the invention provides tinostamustine or a pharmaceutically acceptable salt thereof for use in the treatment of lymphoma or a T-cell malignant disease in a patient, wherein the patient baseline platelet count and the dose of tinostamustine or pharmaceutically acceptable salt thereof is selected from anyone of A1-A10, B1-B10, C1-C10, D1-D10, E1-E10, F1-F10, G1-G10 or H1-H10 as defined in the table above.

In a fourth aspect, the present invention provides tinostamustine or a pharmaceutically acceptable salt thereof for use in the treatment of lymphoma or a T-cell malignant disease in a patient, wherein the patient has a baseline platelet count of less than or equal to 100×10⁹/L and the tinostamustine or pharmaceutically acceptable salt thereof is administered at a dose of 54 mg/m² or lower (for example 45-54 mg/m² e.g. 50 mg/m²) based on free tinostamustine and the patient's body surface area.

In particular, the present invention provides tinostamustine or a pharmaceutically acceptable salt thereof for use in the treatment of lymphoma or a T-cell malignant disease in a patient, wherein the patient has a baseline platelet count of less than or equal to 100×10⁹/L and the tinostamustine or pharmaceutically acceptable salt thereof is administered at a dose of 50 mg/m² based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count of less than or equal to a second value which is:
70×10⁹/L
75×10⁹/L;
80×10⁹/L;
85×10⁹/L;
90×10⁹/L;
95×10⁹/L;
105×10⁹/L;
110×10⁹/L;
115×10⁹/L; or
120×10⁹/L;
   and the tinostamustine or pharmaceutically acceptable salt thereof is administered at a dose of 54 mg/m² or lower (for example 45-54 mg/m² e.g. 50 mg/m²) based on free tinostamustine and the patient's body surface area.

In other particular embodiments, the invention provides tinostamustine or a pharmaceutically acceptable salt thereof for use in the treatment of lymphoma or a T-cell malignant disease in a patient, wherein the patient has a baseline platelet count of less than or equal to a second value which is 100×10⁹/L and the tinostamustine or pharmaceutically acceptable salt thereof is administered at a dose of:
30 mg/m² based on free tinostamustine and the patient's body surface area;
35 mg/m² based on free tinostamustine and the patient's body surface area;
40 mg/m² based on free tinostamustine and the patient's body surface area;
45 mg/m² based on free tinostamustine and the patient's body surface area;
55 mg/m² based on free tinostamustine and the patient's body surface area;
60 mg/m² based on free tinostamustine and the patient's body surface area;
65 mg/m² based on free tinostamustine and the patient's body surface area; or
70 mg/m² based on free tinostamustine and the patient's body surface area.

Other possible combinations of platelet levels and doses are as follows:

| | | Dose based on free tinostamustine (mg/m²) | | | | | | | |
|---|---|---|---|---|---|---|---|---|---|
| | | 30-40 | 35-45 | 40-50 | 45-55 | 50-60 | 55-65 | 60-70 | 65-75 |
| Platelet | 70 | A1 | B1 | C1 | D1 | E1 | F1 | G1 | H1 |
| level | 75 | A2 | B2 | C2 | D2 | E2 | F2 | G2 | H2 |
| equal to | 80 | A3 | B3 | C3 | D3 | E3 | F3 | G3 | H3 |
| or less | 85 | A4 | B4 | C4 | D4 | E4 | F4 | G4 | H4 |
| than | 90 | A5 | B5 | C5 | D5 | E5 | F5 | G5 | H5 |
| (×10⁹/L) | 95 | A6 | B6 | C6 | D6 | E6 | F6 | G6 | H6 |
| | 100 | A7 | B7 | C7 | D7 | E7 | F7 | G7 | H7 |
| | 105 | A8 | B8 | C8 | D8 | E8 | F8 | G8 | H8 |
| | 110 | A9 | B9 | C9 | D9 | E9 | F9 | G9 | H9 |
| | 115 | A10 | B10 | C10 | D10 | E10 | F10 | G10 | H10 |
| | 120 | A11 | B11 | C11 | D11 | E11 | F11 | G11 | H11 |

In other particular embodiments, the invention provides tinostamustine or a pharmaceutically acceptable salt thereof for use in the treatment of lymphoma or a T-cell malignant disease in a patient, wherein the patient baseline platelet count and the dose of tinostamustine or pharmaceutically acceptable salt thereof is selected from anyone of A1-A11, B1-B11, C1-C11, D1-D11, E1-E11, F1-F11, G1-G11 or H1-H11 as defined in the table above.

In general, the tinostamustine or a pharmaceutically acceptable salt thereof is administered over multiple treatment cycles e.g. 4 to 8 treatment cycles.

In other words, the tinostamustine or a pharmaceutically acceptable salt thereof is administered to the patient and then the patient has a rest period in which no treatment is administered. Each period in which tinostamustine or a pharmaceutically acceptable salt thereof is administered is a treatment cycle.

In one embodiment, the treatment cycle is 21 days i.e. three weeks. In particular, tinostamustine or a pharmaceutically acceptable salt thereof is administered on day 1 of a 21-day treatment cycle.

As mentioned, the invention is based on the surprising finding that the maximum tolerated dose of tinostamustine can be determined by measuring the patient's platelet count before administration. In the embodiment in which the tinostamustine or a pharmaceutically acceptable salt thereof is administered over multiple treatment cycles, the platelet count can be measured before each treatment cycle, to determine the appropriate dose for that cycle.

Accordingly, in one embodiment, the invention provides tinostamustine or a pharmaceutically acceptable salt thereof for use in the treatment of lymphoma or a T-cell malignant disease in a patient, wherein the tinostamustine or a pharmaceutically acceptable salt thereof is administered over multiple treatment cycles and the treatment involves:
(a) determining the patient's platelet count before each subsequent treatment cycle; and
(b) (i) if the patient's initial platelet count was greater than or equal to 200×10⁹/L and the patient's platelet count before the subsequent treatment cycle is greater than or equal to 50×10⁹/L, then the amount of tinostamustine or pharmaceutically acceptable salt thereof administered in the subsequent treatment cycle is 95 mg/m² or greater (for example 95-105 mg/m² e.g. 100 mg/m²) based on free tinostamustine and the patient's body surface area; and
   (ii) if the patient's initial platelet count was greater than or equal to 200×10⁹/L and the patient's platelet count before the subsequent treatment cycle is less than 50×10⁹/L, then the amount of tinostamustine or pharmaceutically acceptable salt thereof administered in the subsequent treatment cycle is 84 mg/m² or lower (for example 75-84 mg/m² e.g. 80 mg/m²) based on free tinostamustine and the patient's body surface area; and;
(iii) if the patient's initial platelet count is between 100×10⁹/L and 200×10⁹/L and the patient's platelet count before the subsequent treatment cycle is greater than or equal to 50×10⁹/L, then the amount of tinostamustine or pharmaceutically acceptable salt thereof administered in the subsequent treatment cycle is 75-85 mg/m² (for example 80 mg/m²) based on free tinostamustine and the patient's body surface area; and
(iv) if the patient's initial platelet count is between 100×10⁹/L and 200×10⁹/L and the patient's platelet count before the subsequent treatment cycle is less than 50×10⁹/L, then the amount of tinostamustine or pharmaceutically acceptable salt thereof administered in the subsequent treatment cycle is from 55-65 mg/m² (for example 60 mg/m²) based on free tinostamustine and the patient's body surface area; and
(v) if the patient's initial platelet count was less than 100×10⁹/L and the patient's platelet count before the subsequent treatment cycle is greater than or equal to 50×10⁹/L, then the amount of tinostamustine or pharmaceutically acceptable salt thereof administered in the subsequent treatment cycle is 54 mg/m² or lower (for example 45-54 mg/m² e.g. 50 mg/m²) based on free tinostamustine and the patient's body surface area; and
(vi) if the patient's initial platelet count was less than 100×10⁹/L and the patient's platelet count before the subsequent treatment cycle is less than 50×10⁹/L, then the amount of tinostamustine or pharmaceutically acceptable salt thereof administered in the subsequent treatment cycle is 44 mg/m² or lower (for example 35-44 mg/m² e.g. 40 mg/m²) based on free tinostamustine and the patient's body surface area.

The tinostamustine or a pharmaceutically acceptable salt thereof for use can be administered by any standard method. However, preferably the tinostamustine or pharmaceutically acceptable salt thereof is administered intravenously. The skilled person is aware as part of the common general knowledge of how to prepare solutions of tinostamustine or its salts for intravenous administration.

In particular, the tinostamustine or pharmaceutically acceptable salt thereof is administered over from 45-75 minutes, for example 50-70 minutes or 55-65 minutes e.g. 60 minutes.

In a fifth aspect, the present invention provides tinostamustine or a pharmaceutically acceptable salt thereof for use in the treatment of lymphoma or a T-cell malignant disease in a patient, wherein the tinostamustine or pharmaceutically acceptable salt thereof is administered:
intravenously;
at a dose of from 20-100 mg/m² based on free tinostamustine and the patient's body surface area, for example 95-105 mg/m², 75-85 mg/m², 55-65 mg/m², 45-55 mg/m² or 35-45 mg/m² (e.g. 100 mg/m²; 80 mg/m², 60 mg/m², 50 mg/m² or 40 mg/m²); and
over a period of time which is 45-75 minutes, for example 50-70 minutes or 55-65 minutes e.g. 60 minutes.

The tinostamustine can be used as free tinostamustine i.e. not as a pharmaceutically acceptable salt.

Alternatively, the tinostamustine can be used in the form of a pharmaceutically acceptable salt which is the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate.

In particular, the tinostamustine is the acetate i.e the salt formed by reacting tinostamustine with acetic acid.

Tinostamustine and its salts can be prepared using methods which form part of the common general knowledge. In particular, reference is made to Example 6 of WO 2010/085377.

The tinostamustine or a pharmaceutically acceptable salt thereof is for use in the treatment of lymphoma.

In particular, the lymphoma is relapsed or refractory lymphoma.

Refractory lymphoma is disease that has not responded to initial treatment. Refractory disease may be disease that is getting worse or staying the same. Relapsed lymphoma is disease that has responded to treatment but then returned.

In particular, the tinostamustine or a pharmaceutically acceptable salt thereof is for use in the treatment of lymphoma which is:
Hodgkin lymphoma; or
non-Hodgkin lymphoma, for example NK/T-cell lymphoma (non-Hodgkin lymphoma that develops from natural killer cells or T lymphocytes), peripheral T-cell lymphoma (PTCLcutaneous T-cell lymphoma (CTCL) or anaplastic large cell lymphoma (ALCL).

In particular, the tinostamustine or a pharmaceutically acceptable salt thereof is for use in the treatment of a T-cell malignant disease which is T-cell-prolymphocytic leukemia (T-PLL).

The tinostamustine or a pharmaceutically acceptable salt thereof can be used as a monotherapy i.e. as the only therapeutic intervention.

Alternatively, the tinostamustine or the pharmaceutically acceptable salt thereof can be used in combination with one or more other compounds or therapies.

If the tinostamustine or pharmaceutically acceptable salt thereof is used in combination with one or more other compounds or therapies are administered concurrently, sequentially or separately.

In one embodiment the one or more other compounds are proteasome inhibitors, for example bortezomib, carfilzomib, marizomib, delanzomib (CEP-18770), oprozomib (ONX 0912), ixazomib (MLN-9708) or LU-102, and preferably bortezomib, carfilzomib and LU-102.

In particular, the one or more other compounds are glucocorticoids, for example dexamethasone, fluocinolone acetonide or prednisone e.g. dexamethasone.

In addition, the tinostamustine or a pharmaceutically acceptable salt can be used in combination with radiotherapy, for example wherein the radiotherapy is given at a dose of 1 to 5 Gy over 5-10 consecutive days, and preferably at 2 Gy over 5-10 consecutive days.

### EXAMPLES

A phase 1 trial was carried out including a dose escalation study to investigate the safety, pharmacokinetic (PK) profiles and efficacy of tinostamustine (EDO-S101) in relapsed/refractory hematologic malignancies.

The patient had relapsed/refractory hematologic malignancies for which there are no available therapies.

### Dose Escalation Levels

| | | Administration | |
|---|---|---|---|
| Level | Dose | time (infusion) | Schedule |
| 1 | 20 mg/m² | 1 hour | Every 21 days |
| 2 | 40 mg/m² | 1 hour | Every 21 days |
| 3 | 60 mg/m² | 1 hour | Every 21 days |
| 4 | 80 mg/m² | 1 hour | Every 21 days |
| 5 | 100 mg/m² | 1 hour | Every 21 days |
| 6 | 120 mg/m² | 1 hour | Every 21 days |
| 7 | One dose level below MTD | 45 minutes | Every 21 days |
| 8 | Two dose level below MTD | 30 minutes | Every 21 days |
| 9 | Escalation until | 30 minutes | Every 21 days |
| | maximum administered | | |
| | dose (MAD) or 150 mg/m² | | |

The assessment of dose-limiting toxicities (DLTs) was based on cycle 1 events alone. Toxicities were assessed regarding type and severity using the National Cancer Institute (NCI) Common Terminology Criteria for Adverse Events (CTCAE) version 4.03, June 2010. Toxicity data were collected for all patients throughout their time on study. Infusion site reactions were assessed using the Phlebitis Scale developed by the Infusion Nurses Society (2011).

DLTs, which were at least possibly related to study drug, were defined as:
Any Grade 3 or 4 non-hematologic toxicity (excluding alopecia and easily correctable electrolyte abnormalities);
Nausea, vomiting or diarrhoea that persists beyond 10 days despite aggressive symptomatic treatment;
Grade 4 neutropenia or thrombocytopenia lasting for 7 days or more;
Any grade 2 or more toxicity, but persist for over 3 weeks; and
Any toxicity resulting in a delay of the next dose administration (cycle 2 day 1≥14).

To ensure patients' safety, the stopping rules were implemented to the dose escalation stage of the study. If the following toxicity, which at least possibly related to the study drug, was observed in 66% or more patients treated at any dose level, the enrolment would have been put on hold to allow the sponsor to assess and address the risk:
Grade 2 increase of serum bilirubin level (>1.5-3.0×the upper limit of normal (ULN));
Grade 2 increase of serum creatinine (>1.5-3.0×baseline; >1.5-3.0×ULN); and
Grade 2 nervous system disorders excluding headache.

Other safety assessments included physical examinations, Eastern Cooperative Oncology Group (ECOG) performance status determinations, electrocardiograms (ECGs), pregnancy testing for women of childbearing potential, documentation of treatment-emergent adverse events (TEAEs), clinical laboratory evaluations including haematology, blood chemistry and urinalysis, vital signs, and documentation of concomitant medication usage.

### PK Assessment

Plasma samples were collected to determine the concentrations of tinostamustine and its metabolites (labelled M2 and M8) by a method fully validated according to the relevant guidelines. The PK profiles of tinostamustine in plasma were assessed in each patient in the escalation stage of the study, in cycle 1 only.

### Tumour Assessment

Response to treatment included evaluation of overall response rate (patients with a CR plus patients with a PR), clinical benefit (CB) rate (patients with CR plus patients with PR plus patients with stable disease (SD)), progression free survival, and overall survival according to the relevant response criteria.

### Independent Data Monitoring Committee (IDMC)

A Data Safety Monitoring Committee (DSMC) was established for this study. It consisted of four members, two independent haematologist-oncologists experienced in clinical trials, (one being nominated as chairman), a physician nominated by the sponsor and a statistician. The decisions to escalate to the next dose level occurred after each cohort patients' data were discussed with the DSMC.

### Dose Escalation Stage Summary

The study had a 3+3 design, in patients with relapsed/refractory hematological malignancies (HM). In these studies, three patients are initially enrolled into a given dose cohort. If there is no DLT observed in any of these subjects, the trial proceeds to enroll additional subjects into the next higher dose cohort. If one subject develops a DLT at a specific dose, an additional three subjects are enrolled into that same dose cohort. Development of DLTs in more than 1 of 6 subjects in a specific dose cohort suggests that the MTD has been exceeded, and further dose escalation is not pursued.

The dose escalation phase included 46 patients enrolled across nine different cohorts. Among all 46 patients, 31 (69%) have discontinued from the study, with the reason for discontinuation being progressive disease (PD) for 17 (38%) patients, adverse events (AE) for 13 (29%) patients, and 1 (2%) patient discontinued from the study because of an “other” reason.

| | | Dosing Cohort: mg/m²/infusion time (mins) | | | | | | | | |
|---|---|---|---|---|---|---|---|---|---|---|
| Disease | N | 20/60 | 40/60 | 60/60 | 80/60 | 100/60 | 120/60 | 80/45 | 60/30 | 80/30 |
| Multiple Myeloma | 19 | 0 | 2 | 1 | 1 | 7 | 0 | 5 | 3 | 0 |
| Non-Hodgkin | 17 | 3 | 0 | 2 | 2 | 1 | 2 | 3 | 4 | 0 |
| lymphoma | | | | | | | | | | |
| Hodgkin lymphoma | 10 | 0 | 1 | 0 | 0 | 0 | 4 | 2 | 2 | 1 |
| Total | 46 | 3 | 3 | 3 | 3 | 8 | 6 | 10 | 9 | 1 |

Six cycles of treatment were planned at each dose level. Patients who benefited from treatment were allowed to receive more cycles. Patients who did not respond to therapy and those who developed early toxicity were treated with one to three cycles.

Of the 46 patients enrolled, 27 of the patients have lymphoma (Hodgkin and non-Hodgkin) and 19 patients have multiple myeloma (MM).

Initially, stage 1 of the study enrolled 20 patients with HM, including 9 patients with lymphoma and 11 patients with MM, into a total of 5 ascending dose cohorts as follows:
20 mg/m² over 1 hour (HM20/60 cohort; N=3)
40 mg/m² over 1 hour (HM40/60 cohort; N=3)
60 mg/m² over 1 hour (HM60/60 cohort; N=3)
80 mg/m² over 1 hour (HM80/60 cohort; N=3)
100 mg/m² over 1 hour (HM100/60 cohort; N=8)

The dose of 100 mg/m² administered over 60 minutes was determined to be the maximum tolerated dose (MTD) in the subset of patients with MM (N=11). Although no dose-limiting toxicities (DLTs) occurred in cycle (C) 1 in this dose level, three patients experienced Grade 3 and 4 thrombocytopenia in C2 and C3, leading to the patients' withdrawal from the study.

Subsequently, cohorts with tinostamustine at lower doses administered over shorter infusion times of 45 minutes and 30 minutes were initiated in patients with MM. A total of 8 patients with MM were enrolled in 2 cohorts, as follows:
60 mg/m² over 30 minutes (MM60/35 cohort; N=3)
80 mg/m² over 45 minutes (MM80/45 cohort; N=5)

Patients with lymphoma, as per protocol, were enrolled in a separate cohort with tinostamustine 120 mg/m² administered over 1 hour. Two out of six patients experienced DLTs. Consequently, 120 mg/m² administered over 1 hour was determined as the MAD for the treatment of lymphoma subpopulation and the dose of 100 mg/m² determined as the MTD. Tinostamustine at lower doses administered over shorter infusion times of 45 and 30 minutes were initiated in patients with lymphoma. A total of 18 patients with lymphoma were enrolled in 4 lymphoma cohorts, as follows:
120 mg/m² over 1 hour (LYM120/60 cohort; N=6)
80 mg/m² over 45 minutes (LYM80/45 cohort; N=5)
60 mg/m² over 30 minutes (LYM60/30 cohort; N=6)
80 mg/m² over 30 minutes (LYM80/30 cohort; N=1)

The recruitment to the last cohorts of 80 mg/m² over 30 minutes was stopped due to the high Cₘₐₓ. High Cₘₐₓ and increased hematotoxicity was also observed in the phase ½ solid tumour study with tinostamustine in 30 minutes infusion time. Therefore, the one hour infusion time was selected, and the determination of MTD for shorter infusion was terminated.

### Safety

### Overview

Among all 46 patients, 42 (91%) experienced at least one treatment-emergent adverse event (TEAE), with at least one TEAE considered by the investigator to be study drug-related for 38 patients. The most common types of TEAEs were blood and lymphatic system disorders (25 patients; 54%), gastrointestinal (GI) disorders (24 patients; 52%), and general disorders and administration site conditions (23 patients; 50%).

Overall, the most common individual TEAEs were hematologic abnormalities, including thrombocytopenia/platelet count decreased (24 patients (52%)), anaemia (18 patients (39%)), nausea and neutropenia/neutrophil count decreased (each 13 patients (29%)) and leukopenia/white blood cell count (WBC) decreased (10 patients (21%)). All other TEAEs occurred in <20% of patients.

Overall, 29 (63%) patients experienced a grade 3 or 4 TEAE, with 60% (28 patients) and 28% (13 patients) experiencing at least 1 grade 3 and grade 4 TEAE, respectively. No patient experienced a TEAE with an outcome of death (i.e., a grade 5 TEAE). The most common individual grade 3/4 TEAEs were hematologic abnormalities, including thrombocytopenia (18 patients (39%)), neutropenia/neutrophil count decreased (13 patients (29%)), anemia (10 patients (22%)), leukopenia/WBC decreased (seven patients (15%)), and lymphopenia (4 patients (8.6%)). All other grade 3/4 TEAEs occurred in one or two patients only.

Infusion-site phlebitis events were not common, with only one (2%) patient experiencing this type of event. Eight (18%) patients experienced at least 1 serious adverse event (SAE), including febrile neutropenia and sepsis (each 2 patients (2%)) and dyspnea, hypersensitivity, osteomyelitis, pleural effusion, pneumonia, and thrombocytopenia (each one patient (2%)). Of these events, dyspnea and pleural effusion were considered by the investigator to be unrelated to study drug. The remaining events were considered study drug-related.

11 (23%) patients discontinued study drug because of a TEAE. The only TEAEs leading to study drug discontinuation for >1 patient were thrombocytopenia (7 patients (15%)) and neutropenia (two patients (4%)). All other TEAEs leading to study drug discontinuation were reported for one (2%) patient only and included anaphylactic reaction, febrile neutropenia, hypersensitivity, pleural effusion, and pruritus. A summary of the most common (overall incidence >10%) TEAEs overall, by dose regimen, is as follows, wherein patients are counted if they suffered at least one of the class of TEAE (using the MedDRA preferred term):

| | Patients |
|---|---|
| Adverse event | (N = 46) |
| TEAE | 42 (91.3) |
| Treatment-related* TEAE | 38 (82.6) |
| Grade 3 TEAE | 28 (60.8) |
| Grade 4 TEAE | 13 (28.2) |
| Infusion site phlebitis | 1 (2.1) |
| SAE | 8 (17.4) |
| TEAE leading to early study discontinuation | 11 (23.9) |
| TEAE with outcome of death | 0 (0) |

*Treatment-related TEAE are AEs recorded as relationship possible, probable or definite.

### Haematoloqy

The principle toxicities associated with tinostamustine have been hematologic abnormalities, primarily thrombocytopenia/platelet count decreased (hereafter referred to as thrombocytopenia), anaemia, neutropenia/neutrophil count decreased (hereafter referred to as neutropenia), and leukopeniaNBC decreased (hereafter referred to as leukopenia). Across cohorts 20/60 to 120/60, a dose relationship was apparent with regard to the incidence of these common hematologic abnormalities, with the incidence increasing with increasing dose.

### Thrombocytopenia

Overall, 52% (24/46) of patients experienced at least 1 incidence of thrombocytopenia. Across cohorts 20/60 to 120/60, the incidence of thrombocytopenia generally increased with increasing dose. Thrombocytopenia was also common when tinostamustine was administered over a shorter infusion period, with an incidence of 47% (9/19) across all cohorts at which tinostamustine was administered over 30 or 45 minutes. When tinostamustine was administered over 60 minutes, the incidence of thrombocytopenia was 58% (15/26). Thrombocytopenia was grade 3 or 4 in intensity for 18 of these 24 patients (incidence 40% overall). Again, across cohorts 20/60 to 120/60, a dose relationship was apparent with regard to the incidence of grade 3/4 thrombocytopenia, with an incidence of 71% (7/14) at doses of 100 or 120 mg/m² versus 8% (1/12) at doses of 20 to 80 mg/m². For one (2%) patient (HM120/60 cohort), thrombocytopenia (grade 3) was serious and also led to study discontinuation. All other cases of thrombocytopenia were non-serious. Thrombocytopenia was generally persistent. In total, thrombocytopenia led to study drug and/or study discontinuation for 9 (19%) patients, 7 of whom received tinostamustine at a dose of 100 or 120 mg/m² over 60 minutes, making it the most common TEAE leading to discontinuation.

### Anaemia

Overall, 18 (39%) patients experienced at least one incidence of anaemia. Across cohorts 20/60 to 120/60, the incidence of anaemia generally increased with increasing dose, with an incidence of 64% (9/14) at doses of 100 or 120 mg/m² versus 25% (3/12) at doses of 20 to 80 mg/m². Mitigation of anaemia was not apparent with lower tinostamustine doses over a shorter infusion time, with an incidence of 37% (7/19) across all cohorts at which tinostamustine was administered over 30 or 45 minutes. Anaemia was grade 3 or 4 in intensity for 10 (22%) patients overall, again, with a higher incidence at doses of 100 or 120 mg/m² (36% (5/14)) than at doses of 20 to 80 mg/m² (8% (1/12)). All cases of anaemia were non-serious, and none led to study or study drug discontinuation.

### Neutropenia

Overall, 13 (28%) patients experienced at least one incidence of neutropenia, with all 13 patients experiencing grade 3 or 4 neutropenia. Across cohorts 20/60 to 120/60, the incidence of neutropenia was higher at doses of 100 or 120 mg/m² (50% (7/14)) than at doses of 20 to 80 mg/m² (25% (3/12)). Neutropenia was less common with tinostamustine was administered over shorter infusion times (16% (3/19)). All cases of neutropenia were non-serious. Two patients discontinued study drug and/or the study because of neutropenia.

### Leukopenia

Overall, 10 (22%) patients experienced at least one incidence of leukopenia, with at least one such event being grade 3 or 4 in intensity for 7 (16%) patients. Across cohorts 20/60 to 120/60, the incidence of leukopenia was higher at the higher doses of 100 or 120 mg/m² (36% (5/14)) than at doses of 20 to 80 mg/m² (17% (2/12)). The incidence of leukopenia was 16% (3/19) when tinostamustine was administered over 30 or 45 minutes compared to 27% (7/26) when administered over 60 minutes. No patient discontinued study drug or the study because of leukopenia.

### Biochemistry

Treatment-emergent shifts to grade 3 or 4 clinical chemistry abnormalities were not common. Only one (2%) patient with normal or grade 1 or 2 values at baseline experienced a treatment-emergent grade 4 clinical chemistry abnormality, hyperuricemia. One (2%) patient each with normal or grade 1 or 2 values at baseline experienced a treatment-emergent shift to grade 3 aspartate aminotransferase increased and grade 3 hypoalbuminemia. Clinical chemistry TEAEs are summarized as follows (using MedDRA preferred term):

| | All Patients |
|---|---|
| | (N = 46) |
| MedDRA preferred term | n (%) |
| Blood potassium | 5 (10.8) |
| decreased/hypokalaemia | |
| Blood creatinine increased | 4 (8.6) |
| C-reactive protein increased | 2 (4.3) |
| Blood alkaline phosphatase increased | 1 (2.1) |
| Blood magnesium decreased | 1 (2.1) |
| Hyperuricemia | 1 (2.1) |
| Hyperglycaemia | 1 (2.1) |

Individual clinical chemistry TEAEs were also not common. The only clinical chemistry TEAEs occurring in >1 patient were blood potassium decreased/hypokalemia (5 patients (11%)) and blood creatinine increased (4 patients (9%)). In addition, two (4%) patients experienced C-reactive protein increased. All but one clinical chemistry TEAE were non-severe. One (2%) patient experienced grade 3 hyperuricemia, with this event considered by the investigator to be unrelated to study drug.

Given the low overall incidence of clinical chemistry abnormalities, any dose relationship could not be ascertained.

### DLTs and MTD

DLTs occurred in the dose cohort of 120/60, in the following patients:
Grade 4 thrombocytopenia lasting for 7 days or more; and
Prolonged thrombocytopenia/toxicity resulting in the delay of the next dose administration (C2 day 1≥14 days)

The dose of 100 mg/m² was determined as MTD for lymphoma and multiple myeloma patients

### Cardiac Safety

No patient had a treatment-emergent ECG abnormality that was indicated to be clinically significant.

One patient had a treatment-emergent corrected QT interval by Fredericia (QTcF interval)>500 msec.

One subject in the MM 80/45 cohort, who had a screening QTcF of 423 msec, had a prolonged QTcF interval of 677 msec at the discontinuation visit, three weeks after his only study drug dose on day 1 of C1. A non-specific T wave abnormality was evident in the anterior leads at that time. The QTcF interval prolongation was reported as a TEAE (MedDRA preferred the term electrocardiogram QT interval prolonged), with this event assessed by the investigator as grade 2 in intensity and possibly related to study drug. No further follow-up was available. The patient subsequently died as a result of his underlying disease.

No other patient had an ECG abnormality reported as a TEAE.

Overall, the incidence of cardiac disorders was relatively low (11% (5/45)), with the only cardiac disorder reported for >1 patient being tachycardia (2 patients; 4%). Only 1 (2%) patient experienced a cardiac disorder that was considered by the investigator to be study drug-related, grade 1 palpitations. All cardiac disorders were grade 1 or 2 in intensity and non-serious and none led to study drug discontinuation.

### Pharmacokinetics and Pharmacokinetic (PK)/Pharmacodynamic (PD) Safety Modelling

The observed toxicities during the escalation stage of the trial over 20 to 120 mg/m² at infusion times 60, 45 and 30 minutes, which appeared to be critical for dose and schedule, were haematological only. Tinostamustine impacted on peripheral blood lymphocytes, neutrophils and platelets by reducing cell counts over the treatment cycles.

Surprisingly, although lymphocytes were considered the target cell population, the reduction of platelets became dose-limiting.

The key pharmacokinetic parameters, the relationship between the pharmacokinetics and the impact on peripheral blood cells were analyzed. A PK model was developed to describe and predict the dose-haematological toxicity relationship in a wider population.

### Pharmacokinetics (PK)

Patients of all cohorts and over all dose levels were sampled according to the following schedule: 0.5 hours prior to dose administration, and at 15, 30 and 45 minutes and 1, 1.25, 1.50, 2, 3, 8, 24, 48 and 72 hours from the start of tinostamustine infusion. Plasma samples were collected according to the Laboratory Manual for the determination of tinostamustine and metabolites M2 and M8 concentrations using a method fully validated according to the relevant guidelines.

The following PK parameters were assessed either by using a non-compartmental analysis or were obtained through the population PK model:
Cₘₐₓ, AUC, and Tₘₐₓ for each dose cohort and infusion time;
Terminal half-life;
Volumes of distribution;
Linearity and variability over the dose range; and
Impact of shorter infusion time from 60 to 45 and 30 min for a given dose on safety.

The sample analysis showed tinostamustine parent compound being the main active component in plasma and metabolites M2 and M8 only made up for <1% and <10%, respectively. Therefore only the tinostamustine parent compound was considered for PK data for analyses and conclusions. The following table summarizes the main exposure parameters over the studied dose range and infusion time on day one:

| | Infusion | Mean (SD) | | | |
|---|---|---|---|---|---|
| Dose | time | Cₘₐₓ | AUC | Tₘₐₓ | No. |
| (mg/m²) | (minutes) | (ng/mL) | (h · ng/mL) | (min) | patients |
| 20 | 60 | 241 (20) | 193 (30) | 48 (12) | 3 |
| 40 | 60 | 1162 (599) | 932 (425) | 24 (18) | 3 |
| 60 | 30 | 1724 (1416) | 627 (491) | 18 (6) | 9 |
| 60 | 60 | 640 (85) | 592 (75) | 60 (0) | 3 |
| 80 | 45 | 1277 (319) | 794 (151) | 24 (6) | 5 |
| 80 | 60 | 1064 (678) | 902 (653) | 48 (24) | 3 |
| 100 | 60 | 1955 (875) | 1657 (523) | 42 (24) | 6 |
| 120 | 60 | 1773 (507) | 1638 (578) | 48 (12) | 6 |

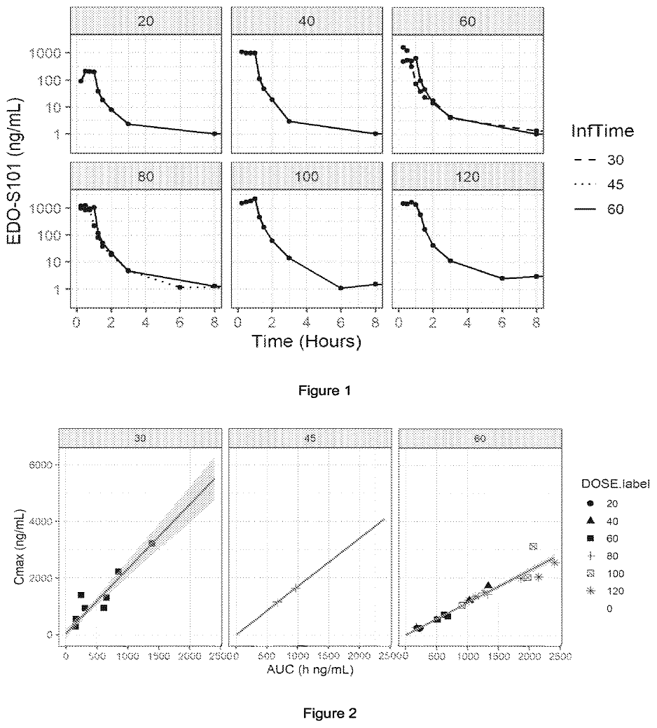

The median PK profiles over a dose range from 20 to 100 mg/m² and at different rates of infusion are shown in FIG. 1.

FIG. 2 shows that the decrease of the infusion time from 60 to 30 minutes doubled the Cₘₐₓ for the same AUC. However, the Cₘₐₓ was variable in particular in the 60 mg/m² over 30 minutes infusion group, where the standard deviation was the largest at 1416 ng/mL. The time to maximum concentrations was achieved at the end of infusion, between 45 and 60 minutes for the 60 minute infusion, and at around 30 minutes for the shorter infusions. Decline from peak concentration occurred in a bi-phasic manner.

A compartmental population PK analysis of tinostamustine was conducted to characterize its concentration-time profiles and its dependency on patient factors. For the analysis, the 24 hours plasma concentrations of tinostamustine at day one from 36 patients was used from whom the data was available. The concentration-time profiles were best described with a two-compartmental model with first order clearance from the central compartment. The model analysis showed that the PK of tinostamustine was dose-linear in the investigated dose range of 20 to 120 mg/m² and for infusion times of 30 to 60 minutes. Further, it was found that the PK did not depend on patient age, body weight or gender. The peripheral volume of distribution was 11.5 L, which was comparable to the interstitial water volume. The between patient variability in the clearance expressed as the coefficient of variation (% CV) was average at 27% and for the central volume of distribution high at 65%, and the large variability in the central volume is likely a result of the observed variability in Cₘₐₓ:

| Parameter | Estimation | |
|---|---|---|
| Central volume of distribution | 20.6 | L (% CV = 65%) |
| Peripheral volume of distribution | 11.5 | L |
| Clearance | 6.75 | L/day (% CV = 27%) |
| α half-life | 27 | minutes |
| B half-life | 3.6 | hours |

### PK/PD Safety Modelling

The obtained exposure parameters (Cₘₐₓ and AUC) and PK profiles from individual patients and the respective haematological laboratory data were collected over the treatment duration and analysed regarding the relationship to peripheral blood cell counts (lymphocytes, neutrophils, platelets).

### Materials and Methods

The effect of tinostamustine on blood cell counts was studied by investigating the relationship between the nadir of the blood cell counts and the tinostamustine nominal dose in mg/m², Cₘₐₓ or AUC₀₋₂₄ hours (predictors). The nadir was defined as the lowest blood cell count observed after the first administration. The nadir was extracted irrespective of the treatment duration, thus, the analysis data set included patients who received only one dose and patients who received multiple treatment cycles with tinostamustine. The baseline blood cell count, which was the count before the first administration, was included in the analysis as the cell count at dose, Cₘₐₓ or AUC equal to 0. Thus, it was assumed that the baseline blood cell count was the effect level without treatment with tinostamustine. With an exploratory analysis all observations each predictor-nadir relationship was fitted with a least-squares method in R. The dose-nadir relationship was further investigated with a non-linear mixed effects approach. The advantage of the non-linear mixed effects approach compared to the exploratory analysis was that different external and patient factors could be investigated at the same time and the between patient variability can be described. The infusion time, type of blood cancer (Hodgkin lymphoma, non-Hodgkin lymphoma, follicular lymphoma (FL), multiple myeloma, or unknown), blood cell count at baseline and the patients' age, bodyweight and sex were tested as factors on the dose-blood cell count nadirs relationship. The effects were tested at a significance level of p=0.01. The dose-nadir relationships were described either with a sigmoid curve:

$$Cell count = Baseline ( 1 - \frac{{D}^{Y}}{EC {50}^{Y} + {D}^{Y}} )$$

or an exponential curve:

$$Cell count = Baseline . {e}^{Dlog ( 2 ) / EC 50}$$

where D was the nominal dose of tinostamustine in mg/m². Based on the above baseline parameters, EC50 were estimated for platelets, neutrophils and lymphocytes for each individual patient assuming that individual parameters would be log-normally distributed. The parameter estimation was done with Monolix2018R1. The analysis included 46 patients for which the treatment information and the blood cell counts were available. Four patients were excluded from the analysis because of disease progression.

The non-linear mixed effects model describing the dose-nadir relationship was used to simulate dose-nadir response curves and to predict the percent patients with grade 3 and grade 4 AE for different doses. For simulations, the patient factors from the patients from study S1001 were used to generate a population via re-sampling of the blood cell counts at baseline. This ensured that the correct correlations between the different blood cell counts where maintained. The 46 patients were resampled 40 times producing a total of 1400 patients from which the statistics was computed. The AE grades used for the analysis were defined as follows:

| AE grade | 2 | 3 | 4 |
|---|---|---|---|
| Platelets (10⁹/L) | 75 | 50 | 25 |
| Neutrophils (10⁹/L) | 1.5 | 1 | 0.5 |
| Lymphocytes (10⁹/L) | 0.8 | 0.5 | 0.2 |

### Pharmacokinetic Metrics and the Relationship to Peripheral Blood Cell Counts

It became apparent through the escalation stage that rising doses of tinostamustine caused a reduction of peripheral blood cell counts, namely lymphocytes, neutrophils and platelets. It was therefore analysed if blood cell compartments had a differential sensitivity to tinostamustine and which of the PK metrics (Cₘₐₓ, AUC or dose) would be best correlated and able to predict effects of various tinostamustine doses.

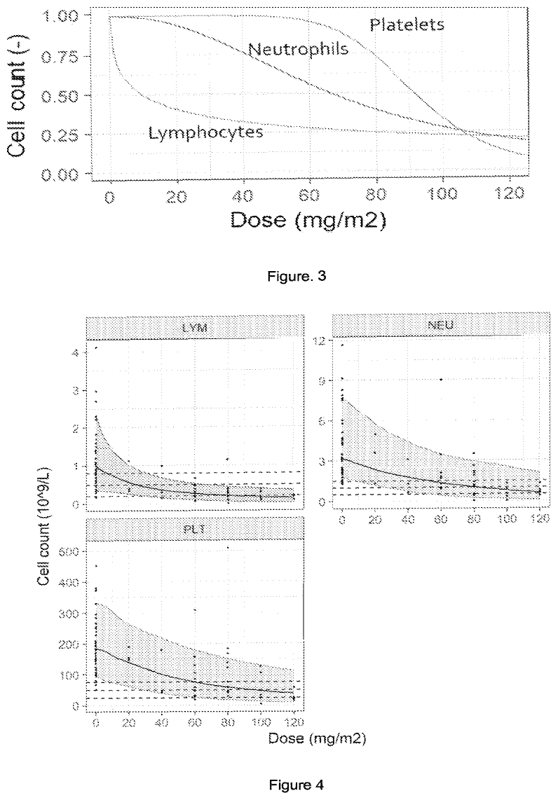

The exploratory analysis confirmed the correlation of rising exposure or doses with the decline in blood cells of all three compartments. There was no significant difference if Cₘₐₓ, AUC or the nominal dose was used but using the dose as the metric, the effects of the dose lead to the best separation of the impact on lymphocytes, neutrophils and platelets, see FIG. 3. The analysis of Cₘₐₓ and AUC versus cell count has not been shown.

The respective nadir data points were fitted with a non-linear mixed effects model to determine the respective EC50 values of tinostamustine doses on peripheral blood cell counts. The data suggested that tinostamustine had an early and profound impact on lymphocytes, followed by neutrophils and finally platelets, see FIG. 4 wherein the dots represent observed blood cell counts, the black line represents predicted median nadir, the shaded area represents the 80th percentile of the predicted nadir, and the dashed lines show grade 2, 3 or 4 AEs. The effects on the lymphocyte compartment were regarded as an effect on the target cell population as the diseases investigated in this trial were all arising from lymphocytes.

Lymphocytes were the most sensitive cell population with an estimated EC50 at a dose of 28.3 mg/m² (dose required to reduce the cell count by 50%). Neutrophils were the second most sensitive with an EC50 of 49.1 mg/m². As reported in the safety section above, there was no increase in infections or a high incidence in neutropenic fever observed. Platelets were the most robust cell compartment with an EC50 of 55.5 mg/m². The decline in platelets was considered dose defining because recovery exceeded the length of the treatment cycle or was a DLT (120 mg/m²). The different sensitivities also translated into different dose levels that would result in a grade 3 AE in 50% of the treated patients. For lymphocytes, a dose of 25 mg/m² was predicted to result in grade 3 AE in 50% of the patients. For neutrophils, the predicted dose level was 80 mg/m² and for platelets, 95 mg/m². The estimated model parameters and predicted dose that would result in a grade 3 AE related to the respective blood cell type in 50% of the patients are as follows:

| | | EC50 | Dose for grade 3 |
|---|---|---|---|
| | Baseline | (mg/m²) | AE (mg/m²) in |
| Cells | (10⁹/L) | (% CV) | 50% of patients |
| Platelets | 192 | 55.5 (72%) | 95 |
| Neutrophils | 3.25 | 49.1 (70%) | 80 |
| Lymphocytes | 1 | 28.3 (40%) | 25 |

### Other Metrics Influencing Peripheral Blood Cell Counts

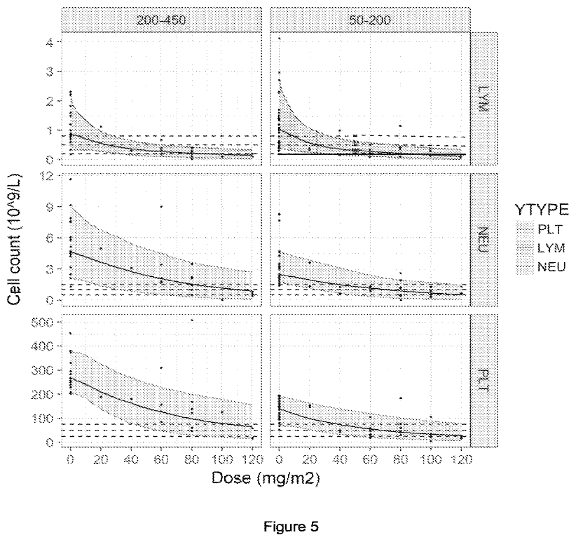

The trial recruited heavily pre-treated and elderly patients, which often based on age and prior therapy have a limited capacity to regenerate peripheral blood cells. The PK, haematology laboratory and demographic patient factors were therefore analyses as to whether there was a relationship to the observed haematological toxicity. The factors taken for analysis were age, gender, and underlying malignancy. The approach was to use a mixed-effects model to identify possible relationships and statistical significant covariates. The results showed no impact of age, sex and underlying disease or infusion time. The one determining factor to determine grade 3 or 4 platelet decrease was the platelet count at the start of therapy. The relation is shown in FIG. 5.

### Dose Tolerability and Platelet Counts at Baseline

As it became apparent that the platelet count at baseline is determining the tolerability of a given dose, we looked at the platelet count at baseline distribution and did further simulations with various cut-off platelet counts in order to understand if there were patient groups who would best be treated with different doses.

The data set for analysis comprised of 42 patients treated over the dose range from 20 to 120 mg/m². Four patients were excluded because disease progression in the bone marrow confounded the effect of tinostamustine and a drug effect on platelet counts could not be established.

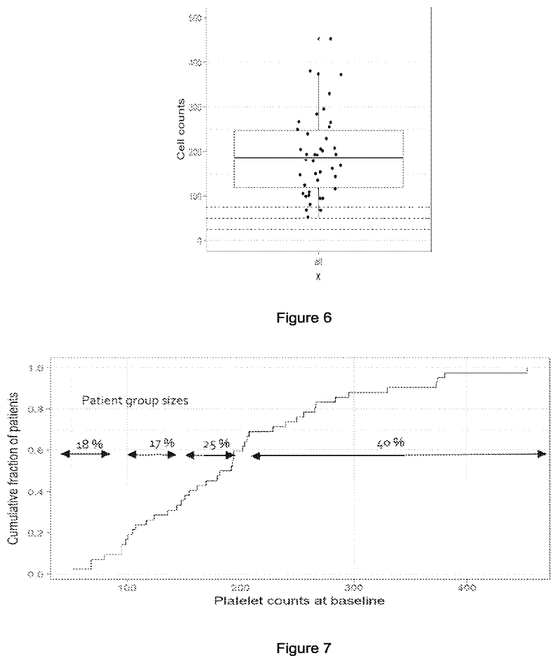

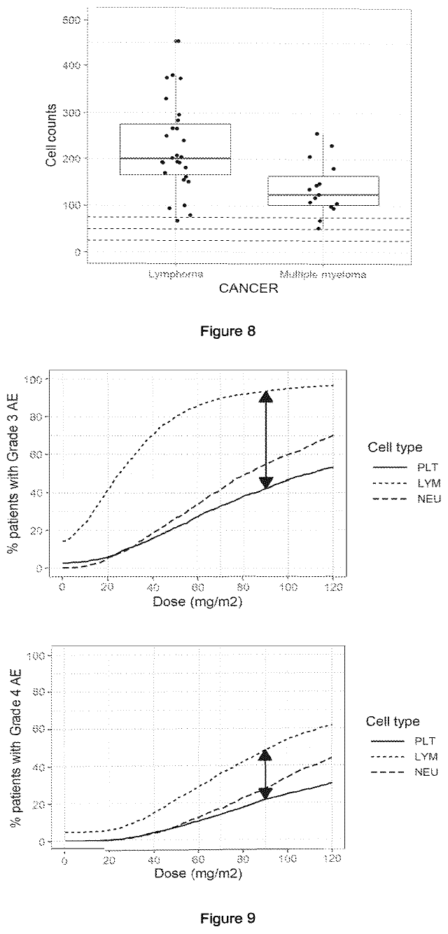

The distribution of platelet counts at baseline is shown in FIG. 6. The median platelet count in the analysis population is 180×10⁹/L and only few patients are in the range of 100×10⁹/L. The cumulative curve for platelet count at baseline in FIG. 7 shows indeed that only 18% of patients are at 100×10⁹/L or lower, a population which was considered most vulnerable to platelet loss.

Platelet counts were also looked at per underlying disease as it was hypothesized that multiple myeloma patients may have entered the study with lower counts because the disease sits in the bone marrow and patients generally had a higher number of previous treatment. The median counts between lymphoma patients (200×10⁹/L) were substantially higher than for patients with multiple myeloma (median 120×10⁹/L). However, the underlying disease is not regarded a relevant factor to determine a tolerable dose.

For the simulations and in order to support the selection of a RP2D, a few assumptions were made. We set the rate of grade 4 thrombocytopenia for a given dose at not higher than 20% and we defined a 90% chance for a grade 3 lymphocytopenia as a surrogate for sufficient efficacy. The reference case was the dose over the entire analysis population (all platelet counts) for these criteria, see FIGS. 9a and b. The reference dose for this scenario was determined to be 90 mg/m².

Simulations were performed against the reference group by dividing the analysis population into two groups with different cut-off counts of platelet counts (×10⁹/L):
Case 1 two groups: 50-100 and 100-450;
Case 2 two groups: 50-150 and 150-450; and
Case 3 two groups: 50-200 and 200-450;
   and into one scenario where we analysed a case where three groups were formed.

Case 4 three groups: 50-100, 100-200 and 200-450.

The simulation by dividing patients into three groups according to their platelet counts at baseline lead to the following results in term of tolerable doses which would represent a 20% risk to experience a grade 4 thrombocytopenia and a 90% chance to have a grade 3 lymphocytopenia:

| | | | | Grade 4 | Grade 4 | Grade 4 LYM | Grade 3 LYM |
|---|---|---|---|---|---|---|---|
| | Groups | Percent | Dose | PLT AE rate | PLT AE rate | AE rate | AE rate |
| Case | (cells 10⁹/L) | patients | (mg/m²) | At 90 mg/m² | At dose | At 90 mg/m² | At dose |
| 4 | 50-100 | 18 | 50 | 45% | 20% | 99% | 95% |
| | 100-200 | 42 | 95 | 20% | 20% | 90% | 90% |
| | 200-450 | 40 | 120+ | 10% | 20% | 95% | 98% |

The simulations revealed that the lowest dose, which can be expected to have a meaningful impact on lymphocytes is 50 mg/m². On the other end, patients with platelet counts above 200×10⁹/L can be treated with the highest dose explored in the escalation stage, namely 120 mg/m². The following observations were also taken into account for the recommendation of cut-off counts and the respective dose:
i. Patients with a platelet count of 100×10⁹/L or lower are the most vulnerable in terms of platelet loss and should be treated with the dose just effective and having the least impact on platelet counts.
ii. Patients with multiple myeloma rarely had platelet counts of more than 200 10⁹/L and therefore did not tolerate a dose of 100 mg/m².
iii. Patients with lymphoma generally had higher platelet counts and 1 patient with refractory HL was treated with 120 m/m² over 6 cycles and achieved a CR.

As a result of the simulations of case 4 and additional observations as above, it is recommended to set the following doses given once every 3 weeks per category of platelet counts at baseline:
i. Dose for platelet counts of 100×10⁹/L or lower is 50 mg/m² over 60 minutes infusion, d1 q3w (i.e. day 1, every third week).
ii. Dose for platelet counts from 100-200×10⁹/L is 80 mg/m² over 60 minutes infusion, d1 q3w.
iii. Dose for platelet counts from 200×10⁹/L or higher is 100 mg/m² over 60 minutes infusion, d1 q3w.

### PK Summary

The PK of tinostamustine over a dose range of 20-120 mg/m² given over 60 minutes infusion time is linear. The main toxicity is haematological with a decline in peripheral blood lymphocytes, neutrophils and platelets. The decline in platelets is dose limiting with either causing a prolonged recovery time exceeding the treatment cycle observed in multiple myeloma patients at 100 mg/m², or as a DLT in cycle 1 in patients with lymphoma (120 mg/m²).

The peripheral cell compartments show a differential sensitivity to tinostamustine with lymphocytes being most sensitive and platelets most robust, but showing the most rapid decline at higher doses.

The further modelling of the dose and thrombocytopenia relationship revealed the sole factor to predict grade 3 or 4 thrombocytopenia was the platelet count at the start of therapy. Other factors such as age, gender, underlying disease or infusion time appeared to have no influence on thrombocytopenia.

Simulations with patient groups and various cut-offs for platelets counts showed that the dose levels to be likely well tolerated seemed to fit best to three patients categories:
Patients with platelet counts of 100×10⁹/L or lower would receive 50 mg/m² over 60 minutes infusion.
Patients with platelet counts of higher than 100 and lower than 200×10⁹/L would receive 80 mg/m² over 60 minutes infusion,
Patients with platelet counts of 200×10⁹/L or higher would receive 100 mg/m² over 60 minutes infusion.
Schedule: i.v. once every 3 weeks

### Signals of Efficacy

### Response for all HM

Among all 46 patients, the overall response rate (ORR) was 28% (13 patients) and the clinical benefit rate (CBR) was 45% (21 patients). The best response, as determined by the investigator, was complete response (CR) for 3 (7%) patients, partial response (PR) for 10 (21%) patients, and stable disease (SD) for 12 (26%) patients. 19 (41%) patients had a best response of PD.

Patients with Lymphoma

Among the 27 patients with lymphoma, the ORR was 40% (11 patients) and the CBR was 62% (15 patients). The best response, as determined by the investigator, was CR for 3 (11%) patients, PR for 8 (30%) patients, and SD for 6 (22%) patients. 10 (37%) patients had a best response of PD.

Although the sample size is small, the ORR and CBR were higher among those patients in the LYM60 mg/m²/30 min cohort and 100% (3/3)) than in the L80 mg/m²/45 min cohort.

Signals of efficacy were observed among the 10 patients with HL, with 70% (7 patients) having a response from SD to CR. One patient who achieved a CR was previously primary refractory, and had displayed no response to chemoradiotherapy, brentuximab vedotin or immune checkpoint inhibitors, and had never been the recipient of autologous stem cell transplantation owing to their primary disease.

Upon achieving CR, this patient was consolidated by receiving an allogeneic haemopoietic stem cell (haplo-transplant) and is graft versus host disease free (GvHD)-free (>20 months) following the last tinostamustine dose.

Patients with Multiple Myeloma (MM)

Among the 19 patients with MM, the ORR was 11% (2 patients) and the CBR was 42% (6 patients). The best response, as determined by the investigator, was CR for 0 patients, PR for 2 (11%) patients, and SD for 6 (31%) patients. 9 (47%) patients had a best response of PD. The CBR was the same in each MM dose cohort.

10 (52%) out of 19 patients with MM had refractory disease or were refractory to previous therapies. Median number of previous therapy lines was 6 (2-13) and median age was 72 (54-83).

Some patients with MM showed a rise in peripheral blood light chain values before day 21 indicating an earlier disease recovery. This would indicate that a once every 3-week schedule is not optimal in controlling MM disease and an administration once every 2 weeks may be more appropriate.

### Summary and Conclusions

### Safety

The principle toxicities associated with tinostamustine have been hematologic abnormalities, primarily thrombocytopenia, neutropenia, anaemia. Across all cohorts, a dose relationship was apparent with regard to the incidence of this toxicity, with the incidence increasing with increasing dose and shortening infusion time. Haematological nadir occurs between day 17 and 23. Thrombocytopenia was determined as the DLT at a dose of 120 mg/m² in lymphoma cohort. In multiple myeloma subpopulation, the decline in platelet count was associated with a prolonged recovery of thrombocytes exceeding the treatment cycle and was observed in patients at 100 mg/m².

### PK and Infusion Time:

The PK of tinostamustine over a dose range of 20 to 120 mg/m² given over 60 minutes infusion time is linear. Time to Cₘₐₓ is achieved between 45 and 60 minutes for the 60 minutes. Decline from peak concentration occurred in a bi-phasic manner.

Evaluation of shorter infusion time, especially a 30 minute infusion showed that the Cₘₐₓ was doubled for the same AUC. The Cₘₐₓ was variable in particular in the 60 mg/m²/30-minute infusion cohort, where the standard deviation was the largest at 1416 ng/mL. The sponsor stopped evaluation of shorter infusion time and decided to use 60 minutes infusion for further development.

### Thrombocytopenia and RP2D

Platelet count at baseline was determined as the sole factor to predict grade 3 or 4 thrombocytopenia. Consequently, the recommended doses of tinostamustine depend on the platelet count at treatment initiation.

### Dosing Schedule

Lymphoma: administration on day 1 in 21-day cycle provided to be well tolerated and efficacious in Lymphoma patients.

Multiple Myeloma: some patients with multiple myeloma showed a rise in peripheral blood light chain values before day 21 indicating an earlier disease recovery. This indicates that a once every 3-week schedule is not optimal in controlling MM disease. Therefore, administration on day 1 and day 15 in a 28-day cycle is recommended. It was also considered that the overall development strategy for multiple myeloma does not include studies as a single agent in multi-refractory disease, but rather would continue with a safe and likely effective dose for combination studies with other approved agents such as a proteasome inhibitor, a CD 38 antibody or a Bcl-2 modifying agent.

### Selection of the Recommended Phase 2 Dose

### Recommended Dose and Schedule of Tinostamustine for the Treatment of Relapse Refractory Lymphoma Patients

1. Baseline platelet count ≥200×10⁹/L: the starting tinostamustine dose is 100 mg/m² over 60 minutes infusion. Administration on day 1 of 21-day cycle. If the platelet count decreases to <50×10⁹/L, the dose should be reduced to 80 mg/m² and maintained for subsequent treatment cycles.
2. Baseline platelet count <200×10⁹/L>100×10⁹/L: the starting tinostamustine dose is 80 mg/m² over 60 minutes infusion. Administration day 1 of 21-day cycle. If the platelet count decreases to <50×10⁹/L the dose should be reduced to 60 mg/m² and maintained for subsequent treatment cycles.
3. Baseline platelet count <100×10⁹/L: the starting tinostamustine dose is 50 mg/m² over 60 minutes infusion. Administration day 1 of 21-day cycle. If the platelet count decreases to <50×10⁹/L, the dose should be reduced to 40 mg/m² and maintained for subsequent treatment cycles.

### Recommended Dose and Schedule of Tinostamustine for the Treatment of Relapse Refractory Multiple Myeloma Patients

1. Baseline platelet count ≥100×10⁹/L: the starting tinostamustine dose is 60 mg/m² over 60 minutes infusion. Administration day 1 and day 15 of 28-day cycle. If the platelet count decreases to <50×10⁹/L, the dose should be reduced to 50 mg/m² and maintained for subsequent treatment cycles.
2. Baseline platelet count ≤100×10⁹/L: the starting tinostamustine dose is 50 mg/m² over 60 minutes infusion. Administration day 1 and day 15 of 28-day cycle. If the platelet count decreases to <50×10⁹/L, the dose should be reduced to 40 mg/m² and maintained for subsequent treatment cycles.

1.-22. (canceled)

23. A method of treating T-cell-prolymphocytic leukemia (T-PLL) in a patient in need thereof, comprising administering to the patient tinostamustine or a pharmaceutically acceptable salt thereof at a first dose of 95 to 105 mg/m² based on free tinostamustine and the patient's body surface area, wherein the patient has been determined to have a baseline platelet count of greater than or equal to 200×10⁹/L prior to the administration of the tinostamustine or pharmaceutically acceptable salt thereof.

24. The method according to claim 23, wherein tinostamustine or pharmaceutically acceptable salt thereof is administered over 4 to 8 treatment cycles.

25. The method according to claim 24, wherein tinostamustine or pharmaceutically acceptable salt thereof is administered on day 1 of a 21-day treatment cycle.

26. The method according to claim 24, comprising:
(a) determining the patient's platelet count before each subsequent treatment cycle; and
(b) administering to the patient an amount of tinostamustine or pharmaceutically acceptable salt thereof, based on the results of the patient's platelet count before said each subsequent treatment cycle, wherein the amount is:
(i) 95 mg/m² or greater, based on free tinostamustine and the patient's body surface area, if the patient's initial platelet count was greater than or equal to 200×10⁹/L and the patient's platelet count before the subsequent treatment cycle is greater than or equal to 50×10⁹/L; or,
(ii) 84 mg/m² or lower, based on free tinostamustine and the patient's body surface area, if the patient's initial platelet count was greater than or equal to 200×10⁹/L and the patient's platelet count before the subsequent treatment cycle is less than 50×10⁹/L.

27. The method according to claim 23, wherein the tinostamustine or pharmaceutically acceptable salt thereof is administered intravenously.

28. The method according to claim 27, wherein the tinostamustine or pharmaceutically acceptable salt thereof is administered over 45-75 minutes.

29. The method according to claim 23, wherein tinostamustine is in the form of a pharmaceutically acceptable salt selected from the group of hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate.

30. The method according to claim 23, wherein tinostamustine is in the form of the free compound.

31. The method according to claim 23, wherein the tinostamustine or the pharmaceutically acceptable salt thereof is used as a monotherapy.

32. The method according to claim 23, wherein the tinostamustine or the pharmaceutically acceptable salt thereof is used in combination with one or more other compounds or therapies.

33. The method according to claim 32, wherein the tinostamustine or the pharmaceutically acceptable salt and the one or more other compounds or therapies are administered concurrently, sequentially or separately.

34. The method according to claim 32, wherein the one or more other compounds are selected from glucocorticoids, dexamethasone, fluocinolone acetonide and prednisone.

35. The method according to claim 32, wherein the one or more other compounds are selected from proteasome inhibitors, bortezomib, carfilzomib, marizomib, delanzomib (CEP-18770), oprozomib (ONX 0912), ixazomib (MLN-9708), and LU-102.

36. The method according to claim 32, wherein the one more other therapies comprises radiotherapy.
